(19) United States
(12) Patent Application Publication
(10) Pub. No.: US 2023/0354850 A1
(43) Pub. Date: Nov. 9, 2023

(54) ANIMAL FEED COMPRISING INSECTS OR INSECT MEAL

(71) Applicant: NOVOZYMES A/S, Bagsvaerd (DK)

(72) Inventor: Eduardo Antonio Della Pia, Lyngby (DK)

(73) Assignee: Novozymes A/S, Bagsvaerd (DK)

(21) Appl. No.: 18/245,143

(22) PCT Filed: Sep. 15, 2021
(86) PCT No.: PCT/EP2021/075266

§ 371 Date: Mar. 13, 2023

(30) Foreign Application Priority Data
Sep. 15, 2020 (EP) 20196174.5

(51) Int. Cl. A23K 10/14; A23K 10/20; A23K 20/147; A23K 50/75; A23J 3/04; A23J 3/34; C12N 9/52

(52) U.S. Cl. CPC A23K 10/14; A23K 10/20; A23K 20/147; A23K 50/75; A23J 3/04; A23J 3/341; C12Y 304/21062; C12N 9/52

(57) ABSTRACT

The present invention relates to an animal feed comprising insects or insect meal and a polypeptide having protease activity. It also relates to a method of degrading an arthropod exoskeleton comprising contacting said exoskeleton with a polypeptide having protease activity. The invention further relates to a method for improving nutritional value of insects or insect meal, comprising contacting the insects or insect meal with a polypeptide having protease activity.

### REFERENCE TO A SEQUENCE LISTING

This application contains a Sequence Listing in computer readable form, which is incorporated herein by reference.

### FIELD OF THE INVENTION

The present invention relates to an animal feed comprising an animal protein source. The present invention also relates to a method of degrading an arthropod exoskeleton and a method for improving nutritional value of insects or insect meal. The present invention further relates to use of a polypeptide having protease activity in an animal feed comprising insects or insect meal.

### BACKGROUND OF THE INVENTION

A 60-70% increase in consumption of animal products is expected by 2050. This increase in the consumption will demand enormous resources, the feed being the most challenging because of the limited availability of natural resources, ongoing climatic changes and food-feed-fuel competition. The costs of conventional feed resources such as soymeal and fishmeal are very high and their availability in the future will be limited. Alternatively, insects grow and reproduce easily, have high feed conversion efficiency, and can be reared on bio-waste streams. One kilogram of insect biomass can be produced from on average 2 kg of feed biomass (Collavo, A., et al, 2005, House cricket small-scale farming. In: Paoletti, M. G. (Ed.), Ecological Implications of Minilivestock: Potential of Insects, Rodents, Frogs and Snails, Science Publishers, New Hampshire, pp. 519-544). Insects can feed on waste biomass and can transform it into high value feed resource.

Some studies have been conducted on evaluation of insects, or insect meals as an ingredient in the diets of some animal species, but this field is in infancy.

### SUMMARY OF THE INVENTION

In one aspect, the present invention provides an animal feed comprising an animal protein source and a polypeptide having protease activity, wherein the animal protein source comprises insects or insect meal.

In another aspect, the present invention provides an animal feed comprising insects or insect meal treated with a polypeptide having protease activity.

In a further aspect, the present invention provides a method of degrading an arthropod exoskeleton comprising contacting said exoskeleton with a polypeptide having protease activity.

In a further aspect, the present invention provides a method for improving nutritional value of insects or insect meal, comprising contacting the insects or insect meal with a polypeptide having protease activity.

In a further aspect, the present invention provides a method of preparing an animal feed comprising insects or insect meal, comprising contacting the insects or insect meal with a polypeptide having protease activity.

In a further aspect, the present invention provides a method for treating insect protein source or carbohydrate source including chitin, comprising the step of adding a polypeptide having protease activity to the insect protein source or carbohydrate source.

In a further aspect, the present invention provides use of a polypeptide having protease activity in an animal feed comprising insects or insect meal.

In a further aspect, the present invention provides use of a polypeptide having protease activity in an animal feed comprising insects or insect meal;

in the preparation of a composition for use in an animal feed comprising insects or insect meal;

in the preparation of an animal feed additive for use in an animal feed comprising insects or insect meal;

for improving nutritional value of an animal feed comprising insects or insect meal;

for increasing digestible and/or soluble nitrogen in an animal feed comprising insects or insect meal;

for increasing the degree of hydrolysis of proteins and/or carbohydrates in animal diets comprising insects or insect meal; and/or

for the treatment of proteins and/or carbohydrates from insects or insect meal.

Chitin is a major constituent of the exoskeleton, or external skeleton, of many arthropods such as insects, spiders, and crustaceans. Exoskeletons made of this firm compound support and protect the delicate soft tissues of these animals, which lack an internal skeleton. Chitin is a polysaccharide, a type of carbohydrate that has a basic structure of a repeating chain of sugar molecules. Surprisingly it was found that polypeptides having protease activity are significantly better than chitinases or glucanases at improving nutritional value of arthropod exoskeleton, including insects or insect meal.

### OVERVIEW OF SEQUENCE LISTING

SEQ ID NO: 1 is the amino acid sequence of the protease derived from Nocardiopsis sp. NRRL 18262.

SEQ ID NO: 2 is the amino acid sequence of the S8 protease from Lysobacter IB-9374.

SEQ ID NO: 3 is the amino acid sequence of the S8 protease from Bacillus horneckiae.

SEQ ID NO: 4 is the amino acid sequence of a variant S8 protease from Bacillus sp TY145 protease.

SEQ ID NO: 5 is the amino acid sequence of the Streptomyces griseus GH18 chitinase.

### DEFINITIONS

Fragment: The term “fragment” means a polypeptide having one or more (several) amino acids deleted from the amino and/or carboxyl terminus of a mature polypeptide; wherein the fragment has protease activity. In one aspect, a fragment contains at least 140 amino acid residues, at least 160 amino acids residues, or at least 180 amino acid residues of SEQ ID NO: 1, the mature polypeptide of SEQ ID NO: 2, SEQ ID NO: 3, or SEQ ID NO: 4.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 188 in the numbering of SEQ ID NO: 1. In a further aspect, the mature polypeptide is amino acids 1-338 in the numbering of SEQ ID NO: 2. In a further aspect, the mature polypeptide is amino acids 1-314 in the numbering of SEQ ID NO: 3. In a further aspect, the mature polypeptide is amino acids 1-311 in the numbering of SEQ ID NO: 4.

Sequence Identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

Variant: The term “variant” means a polypeptide having protease activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding 1, 2, 3 or more amino acids adjacent to an amino acid occupying a position. In one embodiment, the number of amino acid substitutions, deletions and/or insertions introduced into the parent polypeptide is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

### DETAILED DESCRIPTION OF THE INVENTION

In one aspect, the present invention provides an animal feed comprising an animal protein source and a polypeptide having protease activity, wherein the animal protein source comprises insects or insect meal.

In an embodiment, the animal feed optionally further comprises an animal protein source selected from the group consisting of meat meal, bone meal, poultry meal, blood, feather meal and seafood meal; and combinations thereof.

### Polypeptides Having Protease Activity

Polypeptides having protease activity, or proteases, are sometimes also designated peptidases, proteinases, peptide hydrolases, or proteolytic enzymes. Proteases may be of the exo-type that hydrolyse peptides starting at either end thereof, or of the endo-type that act internally in polypeptide chains (endopeptidases). Endopeptidases show activity on N- and C-terminally blocked peptide substrates that are relevant for the specificity of the protease in question.

Proteases are classified on the basis of their catalytic mechanism into the following groups: serine proteases (S), cysteine proteases (C), aspartic proteases (A), metalloproteases (M), and unknown, or as yet unclassified, proteases (U), see Handbook of Proteolytic Enzymes, A. J. Barrett, N. D. Rawlings, J. F. Woessner (eds), Academic Press (1998), in particular the general introduction part.

In one embodiment, the proteases for use according to the invention are acid stable proteases. In another embodiment, the proteases for use according to the invention are serine proteases. Preferred proteases according to the invention are acid stable serine proteases. The term serine protease refers to serine peptidases and their clans as defined in the above Handbook. In the 1998 version of this handbook, serine peptidases and their clans are dealt with in chapters 1-175. Serine proteases may be defined as peptidases in which the catalytic mechanism depends upon the hydroxyl group of a serine residue acting as the nucleophile that attacks the peptide bond. In a preferred embodiment, the polypeptide having protease activity of the present invention is a serine protease. In a more preferred embodiment, the serine protease for use according to the invention is a protease of the S1 family or the S8 family.

For determining whether a given protease is a serine protease, and a family S1 protease, reference is made to the above Handbook and the principles indicated therein. Such determination can be carried out for all types of proteases, be it naturally occurring or wild-type proteases; or genetically engineered or synthetic proteases.

The peptidases of family S1 contain the catalytic triad His, Asp and Ser in that order. Mutation of any of the amino acids of the catalytic triad will result in loss of enzyme activity. The amino acids of the catalytic triad of the S1 protease 1 from Saccharomonospora viridis are probably positions His-32, Asp-56 and Ser-137.

The peptidases of family S8 have a catalytic triad in the order Asp, His and Ser in the sequence.

Suitable proteases include those of bacterial, fungal, plant, viral or animal origin e.g. vegetable or microbial origin. Microbial origin is preferred. Chemically modified or protein engineered mutants are included.

The term “subtilases” refers to a sub-group of serine protease according to Siezen et al., Protein Engng. 4 (1991) 719-737 and Siezen et al. Protein Science 6 (1997) 501-523. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547.

Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146. Pancreatin is a mixture of several digestive enzymes produced by the exocrine cells of the pancreas. In an embodiment of the invention, the polypeptide having protease activity of the present invention is not trypsin/pancreatin.

A further protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 20 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I, Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Axtra® PRO CIBENZA® DP100, Ronozyme® ProAct, Ronozyme® ProAct 360, Verazyme® Vemozyme® P, Proteinase (a broad-range endolytic protease, serine protease), TEV protease®, Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3® , FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Protease activity can be measured using any assay, in which a substrate is employed, that includes peptide bonds relevant for the specificity of the protease in question. Assay-pH and assay-temperature are likewise to be adapted to the protease in question. Examples of assay-pH-values are pH 5, 6, 7, 8, 9, 10, or 11. Examples of assay-temperatures are 30, 35, 37, 40, 45, 50, 55, 60, 65 or 70, 80, 90, or 95° C.

Examples of protease substrates are casein, and pNA-substrates, such as Suc-AAPF-NA (available e. g. from Sigma S7388). The capital letters in this pNA-substrate refers to the one-letter amino acid code. Another example is Protazyme AK (azurine-dyed crosslinked casein prepared as tablets by Megazyme T-PRAK). For pH-activity and pH-stability studies, the pNA-substrate is preferred, whereas for temperature activity studies, the Protazyme AK substrate is preferred.

For the purpose of the present invention, protease activity was determined using assays which are described in in the art, such as the Suc-AAPF-pNA assay, Protazyme AK assay, Suc-AAPX-pNA assay and o-Phthaldialdehyde (OPA). For the Protazyme AK assay, insoluble Protazyme AK (Azurine-Crosslinked Casein) substrate liberates a blue colour when incubated with the protease and the colour is determined as a measurement of protease activity. For the Suc-AAPF-pNA assay, the colourless Suc-AAPF-pNA substrate liberates yellow paranitroaniline when incubated with the protease and the yellow colour is determined as a measurement of protease activity.

In a particular embodiment, the protease for use according to the invention is a microbial protease, the term microbial indicating that the protease is derived from, or originates from a microorganism, or is an analogue, a fragment, a variant, a mutant, or a synthetic protease derived from a microorganism. It may be produced or expressed in the original wild-type microbial strain, in another microbial strain, or in a plant; i. e. the term covers the expression of wild-type, naturally occurring proteases, as well as expression in any host of recombinant, genetically engineered or synthetic proteases.

Examples of microorganisms are bacteria, e. g. bacteria of the phylum Actinobacteria phy. nov., e. g. of class I: Actinobacteria, e. g. of the Subclass V: Actinobacteridae, e. g. of the Order I: Actinomycetales, e. g. of the Suborder XII: Streptosporangineae, e. g. of the Family II: Nocardiopsaceae, e. g. of the Genus I: Nocardiopsis, e. g. Nocardiopsis sp. NRRL 18262, and Nocardiopsis alba; e.g. of the species Bacillus or mutants or variants thereof exhibiting protease activity. This taxonomy is on the basis of Berge's Manual of Systematic Bacteriology, 2nd edition, 2000, Springer (preprint: Road Map to Bergey's).

Further examples of microorganisms are fungi, such as yeast or filamentous fungi.

In one embodiment, the polypeptide having protease activity is a polypeptide having an acid stable protease activity.

In the present context, the term acid-stable means, that the protease activity of the pure protease enzyme, in a dilution corresponding to A₂₈₀=1.0, and following incubation for 2 hours at 37° C. in the following buffer:
100 mM succinic acid, 100 mM HEPES, 100 mM CHES,
100 mM CABS, 1mM CaCl₂, 150 mM KCl, 0.01% Triton®X-100, pH 3.5, is at least 40% of the reference activity, as measured using the assay described in pH-stability assay herein (substrate: Suc-AAPF-pNA, pH 9. 0, 25° C.).

In particular embodiments of the above acid-stability definition, the protease activity is at least 45, 50, 55, 60, 65, 70, 75, 80, 85, 90, 95, or at least 97% of the reference activity.

The term reference activity refers to the protease activity of the same protease, following incubation in pure form, in a dilution corresponding to A₂₈₀=1.0, for 2 hours at 5° C. in the following buffer: 100 mM succinic acid, 100 mM HEPES, 100 mM CHES, 100 mM CABS, 1 mM CaCl₂, 150 mM KCl, 0.01% Triton® X-100, pH 9.0, wherein the activity is determined as described above.

In other words, the method of determining acid-stability comprises the following steps:

a) The protease sample to be tested (in pure form, A₂₈₀=1.0) is divided in two aliquots (I and II);

b) Aliquot I is incubated for 2 hours at 37° C. and pH 3.5;

c) Residual activity of aliquot I is measured (pH 9.0 and 25° C.);

d) Aliquot II is incubated for 2 hours at 5° C. and pH 9.0;

e) Residual activity of aliquot II is measured (pH 9.0 and 25° C.);

f) Percentage residual activity of aliquot I relative to residual activity of aliquot II is calculated.

Alternatively, in the above definition of acid stability, the step b) buffer pH-value may be 1.0, 1.5, 2.0, 2.5, 3.0, 3.1, 3.2, 3.3, or 3.4.

In other alternative embodiments of the above acid stability definition relating to the above alternative step b) buffer pH-values, the residual protease activity as compared to the reference, is at least 5, 10, 15, 20, 25, 30, 35, 40, 45, 50, 55, 60, 65, 70, 75, 80, 85, 90, 95, or at least 97%.

In alternative embodiments, pH values of 6.0, 6.5, 7.0, 7.5, 8.0, or 8.5 can be applied for the step d) buffer.

In the above acid-stability definition, the term A₂₈₀=1.0 means such concentration (dilution) of said pure protease which gives rise to an absorption of 1.0 at 280 nm in a 1 cm path length cuvette relative to a buffer blank.

And in the above acid-stability definition, the term pure protease refers to a sample with a A₂₈₀/A₂₆₀ ratio above or equal to 1.70.

Examples of acid-stable proteases for use according to the invention are

a) the proteases derived from Nocardiopsis sp. NRRL 18262, and Nocardiopsis alba; or

b) proteases of at least 60%, at least 65%, at least 70%, at least 75%, at least 80%, at least 85%, at least 90%, or at least 95%, at least 96%, at least 97%, at least 98%, at least 99% or 100% sequence identity to any of the proteases of (a).

In another particular embodiment, the protease for use according to the invention is thermostable.

The term thermostable means one or more of the following: That the temperature optimum is at least 50° C., 52° C., 54° C., 56° C., 58° C., 60° C., 62° C., 64° C., 66° C., 68° C., or at least 70° C.

In a preferred embodiment, the polypeptide having protease activity is selected from the group consisting of:

(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;

(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and

(c) a fragment of the polypeptide of (a), or (b) that has protease activity.

In a more preferred embodiment, the polypeptide comprises or consists of SEQ ID NO: 1, the mature polypeptide of SEQ ID NO: 2, SEQ ID NO: 3, or SEQ ID NO: 4.

### Animal

The term animal includes all animals. Examples of animals are non-ruminants, and ruminants. In a particular embodiment, the animal is a ruminant animal. Ruminant animals include, for example, animals such as sheep, goats, and cattle, e.g. beef cattle, cows, and young calves. In a particular embodiment, the animal is a non-ruminant animal. Non-ruminant animals include mono-gastric animals, e.g. pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; and fish (including but not limited to salmon, trout, tilapia, catfish and carps); and crustaceans (including but not limited to shrimps and prawns). In a preferred embodiment, the animal is a mono-gastric animal, preferably a pig or poultry.

### Insects

In the present context, insects or insecta (from Latin insectum) are hexapod invertebrates and the largest group within the arthropod phylum. Insects have a chitinous exoskeleton, a three-part body (head, thorax and abdomen), three pairs of jointed legs, compound eyes and one pair of antennae. The term “insects” refers to insects in any development stage, such as adult insects, insect larvae and insect pupae. Preferably, adult insects are used. A large variety of insects and worms can be used. Preferably, edible insects or edible worms are used. More preferably, the insects are flies, bugs, mosquitos, butterflies, moths, cicadas, termites, bees, ants, wasps, beetles, grasshoppers, crickets, or mealworms. In one embodiment, the insects are selected from the group consisting of moths, butterflies; flies; beetles; crickets, and mealworms. In another preferred embodiment, the insects are selected from the group of insect orders consisting of: Blattodea; Orthoptera; Diptera; Lepidoptera, and Coleoptera. More preferably, the insects belong to the species: black soldier fly (Hermetia illucens), house fly (Musca domestica), morio worm (Zophobas Morio), mealworm (Tenebrio Molitor) or cricket (Gryllida). In a preferred embodiment, the insects belong to black soldier fly. In a preferred embodiment, the insects belong to buffalo mealworm. In a preferred embodiment, the insects belong to cricket. The insects and worms are preferably cultivated, e.g. in an insect farm. The cultivation allows to control and reduces the risks associated with diseases of insects and with the toxicity of insect-derived feedstuffs, e.g. due to the presence insecticides, in contrast to insects harvested in the nature.

Insects can be treated by a heating, pressing, grinding, cutting, separation, and/or drying process, after which the product is in processed form known as insect meal. Preferably, the insects or worms are thereby reduced in size in an insect meal. This results in a homogeneous starting material of viscous consistency. The squashing and reducing in size can conveniently be done in a micro-cutter mill, although other suitable techniques can also be used. During this step, the particle size of the insects or worm is preferably less than 1 mm. The particle size can be controlled by selection of a specific knife and plate combination and rotating speed; for example one can use a single or double knife and rotating speed could vary between 1000 and 3000 rpm. A skilled person can find suitable conditions in order to reach a desired particle size. A small particle size is advantageous as it facilitates the enzymatic hydrolysis.

### Animal Protein Source

In an embodiment, the animal protein source of the animal feed comprises insects or insect meal.

In another embodiment, the animal protein source of the animal feed comprises insects or insect meal and optionally further comprises an animal protein source selected from the group consisting of meat meal, bone meal, poultry meal, blood, feather meal and seafood meal. In a further embodiment, the seafood meal can be selected from the group consisting of shellfish, crab, lobster, shrimp meal, or fish. In an embodiment, the animal feed comprises animal protein source in an amount of 0.05-25%.

In one embodiment, the animal protein source comprises insect and/or insect meal and further comprises animal protein selected from blood meal, meat, bone meal, feather meal, shellfish, crab, lobster, shrimp meal, and combinations thereof.

### Vegetable Protein Source

In an embodiment, the animal feed of the present invention may further comprise vegetable protein source. In a preferred embodiment, the vegetable protein source can be selected from the group consisting of soybean, soybean meal, rapeseed, canola meal, sunflower, sunflower seed meal, cottonseed meal, DDGS, faba beans, peas, barley, wheat, rye, oat, maize (corn), rice, triticale, sorghum, palm oil cake. In a preferred embodiment, the vegetable protein source can be selected from the group consisting of soybean, soybean meal, and maize (corn). In a further embodiment, the animal feed comprises vegetable protein source typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0.05-25% meat and bone meal; and/or 0-20% whey.

In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, beet, sugar beet, spinach, quinoa, cabbage and combinations thereof. Other examples of vegetable protein sources are cereals such as barley, wheat, rye, oat, maize (corn), rice, and sorghum.

### Feed or Feed Composition

The term feed or feed composition means any compound, preparation, mixture, or composition suitable for, or intended for intake by an animal. According to the present invention, animal feed or animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg. WO 01/58275 corresponds to US 09/779334 which is hereby incorporated by reference.

An animal feed composition according to the invention has a crude protein content of 50-800 g/kg, and furthermore comprises at least one protease as claimed herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) protease/enzyme preparation may also be added before or during the feed ingredient step. Typically a liquid protease/enzyme preparation is added after the pelleting step. The enzyme may also be incorporated in a feed additive or premix.

The final enzyme concentration in the diet is within the range of 0.01-5000 mg enzyme protein per kg diet, for example in the range of 10-2000 mg enzyme protein per kg animal diet.

The protease should of course be applied in an effective amount, i.e. in an amount adequate for improving protein hydrolysis, protein and amino acid digestibility, and/or improving nutritional value of feed. It is at present contemplated that the enzyme is administered in one or more of the following amounts (dosage ranges): 0.01-5000; 0.1-4000; 1-3000; 10-2000; 50-1000; 100-1000; 150-500; or 200-2000—all these ranges being in mg protease protein per kg feed (ppm). In a preferred embodiment, the enzyme is administered in 1-3000 mg protease protein per kg feed (ppm). In a more preferred embodiment, the enzyme is administered in 1-1000 mg protease protein per kg feed (ppm).

For determining mg protease protein per kg feed, the protease is purified from the feed composition, and the specific activity of the purified protease is determined using a relevant assay (see under protease activity, substrates, and assays). The protease activity of the feed composition as such is also determined using the same assay, and on the basis of these two determinations, the dosage in mg protease protein per kg feed is calculated.

The same principles apply for determining mg protease protein in feed additives. Of course, if a sample is available of the protease used for preparing the feed additive or the feed, the specific activity is determined from this sample (no need to purify the protease from the feed composition or the additive).

A premix can contain, for example, per ton of poultry feed, 50 to 200 g of a propylene glycol solution of the mixture of the active compounds, 20 to 1000 g of an emulsifying agent, 50 to 900 g of cereals and by-products, 20 to 100 g of a proteinic support (milk powder, casein, etc) and 50 to 300 g of a mineral component (expanded silica, feed quality lime, bi-calcium phosphate, etc).

A feed additive or premix as described above is finally added the animal feed composition. It is prepared and added such that the amount of the protease corresponds to an intended addition.

Animal feed compositions or diets have a relatively high content of protein. According to the National Research Council (NRC) publications referred to above, poultry and pig diets can be characterised as indicated in Table B of WO 01/58276.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 as disclosed in Table B of WO 01/58276.

For determining mg protease protein per kg feed, the protease is purified from the feed composition, and the specific activity of the purified protease is determined using a relevant assay (see under protease activity, substrates, and assays). The protease activity of the feed composition as such is also determined using the same assay, and on the basis of these two determinations, the dosage in mg protease protein per kg feed is calculated.

In a particular embodiment, the protease, in the form in which it is added to the feed, or when being included in a feed or feed composition, is well-defined. Well-defined means that the protease preparation is at least 50% pure as determined by Size-exclusion chromatography (see Example 12 of WO 01/58275). In other particular embodiments the protease preparation is at least 60, 70, 80, 85, 88, 90, 92, 94, or at least 95% pure as determined by this method.

A well-defined protease preparation is advantageous. For instance, it is much easier to dose correctly to the feed a protease that is essentially free from interfering or contaminating other proteases or other proteins in general. The term dose correctly refers in particular to the objective of obtaining consistent and constant results, and the capability of optimising dosage based upon the desired effect.

The protease preparation can be (a) added directly to the feed (or used directly in a protein treatment process), or (b) it can be used in the production of one or more intermediate compositions such as feed additives or premixes that is subsequently added to the feed (or used in a treatment process). The degree of purity described above refers to the purity of the original protease preparation, whether used according to (a) or (b) above.

Protease preparations with purities of this order of magnitude are in particular obtainable using recombinant methods of production, whereas they are not so easily obtained and also subject to a much higher batch-to-batch variation when the protease is produced by traditional fermentation methods. Such protease preparation may of course be mixed with other enzymes to obtain a preparation with two or more purified enzymes with different or similar activities.

In an embodiment, the animal feed comprises one or more further enzymes, wherein the further enzymes are selected from the group comprising of amylases; phytases; xylanases; galactanases; alpha-galactosidases; phospholipases; and beta-glucanases; or any mixture thereof. In a further embodiment, the further enzymes are selected from the group comprising of amylases; phytases; xylanases; galactanases; alpha-galactosidases; and beta-glucanases; or any mixture thereof.

In a particular embodiment, the feed of the invention further comprises amylase, for example, an alpha-amylase (EC 3.2.1.1).

Suitable amylases which can be used together with protease of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in W001/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase and Preferenz S100 (from Genencor International Inc./DuPont), Ronozyme® A and RONOZYME® RumiStar™ (DSM Nutritional Products).

In a particular embodiment, the feed of the invention further comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Finase® and Quantum® Blue (AB Enzymes), OptiPhos® (Huvepharma) Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the invention further comprises a xylanase (EC 3.2.1.8). Examples of commercially available xylanases include Ronozyme® WX and Ronozyme® G2 (DSM Nutritional Products), Econase® XT and Barley (AB Vista), Xylathin® (Verenium), Hostazym® X (Huvepharma) and Axtra® XB (Xylanase/beta-glucanase, DuPont).

In a particular embodiment, the composition of the invention further comprises galactanases (EC 3.2.1.89).

In a particular embodiment, the composition of the invention further comprises alpha-galactosidases (EC 3.2.1.22).

In a particular embodiment, the composition of the invention further comprises phospholipases (EC 3.1.1.32, EC 3.1.1.4 or EC 3.1.4.4).

In a particular embodiment, the composition of the invention further comprises beta-glucanases (EC 3.2.1.6).

The incorporation of the composition of feed additives as exemplified herein above to animal feeds can in practice be carried out using a concentrate or a premix. A premix designates a preferably uniform mixture of one or more micro-ingredients with diluent and/or carrier. Premixes are used to facilitate uniform dispersion of micro-ingredients in a larger mix. A premix according to the invention can be added to feed ingredients.

A part from the protease and insects or insects meal, the animal feed of the invention comprises an animal feed additive. The animal feed additives of the invention contain at least one fat-soluble vitamin, and/or at least one water soluble vitamin, and/or at least one trace mineral, and/or at least one macro mineral.

Further, optional, feed-additive ingredients are coloring agents, e.g. carotenoids such as beta-carotene, astaxanthin, canthaxanthin, apoester and lutein; aroma compounds; stabilisers; antimicrobial peptides; polyunsaturated fatty acids (PUFAs); reactive oxygen generating species.

Examples of antimicrobial peptides (AMP's) are CAP18, Leucocin A, Protegrin-1, Thanatin, Defensin, Lactoferrin, Lactoferricin, and Ovispirin such as Novispirin (Robert Lehrer, 2000), Plectasins, and Statins.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Usually fat-and water-soluble vitamins, as well as trace minerals form part of a so-called premix intended for addition to the feed, whereas macro minerals are usually separately added to the feed.

The following are non-exclusive lists of examples of these components:
Examples of fat-soluble vitamins are vitamin A, vitamin D3, vitamin E, and vitamin K, e.g. vitamin K3.
Examples of water-soluble vitamins are vitamin B12, biotin and choline, vitamin B1, vitamin B2, vitamin B6, niacin, folic acid and panthothenate, e.g. Ca-D-panthothenate.
Examples of trace minerals are manganese, zinc, iron, copper, iodine, selenium, and cobalt.
Examples of macro minerals are calcium, phosphorus and sodium.
A feed or feed composition of the invention may also comprise at least one probiotic or direct fed microbial (DFM) optionally together with one or more other enzymes. The direct fed microbial may be a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof, preferably from Bacillus subtilis, Bacillus licheniformis, Bacillus amyloliquefaciens, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, lactobacillus rhamnosus, Clostridium butyricum, Bifidobacterium animalis ssp. animalis, Lactobacillus reuteri, Bacillus cereus, Lactobacillus salivarius ssp. salivarius, Megasphaera elsdenii, Propionibacteria sp and more preferably from Bacillus subtilis strains 3A-P4 (PTA-6506); 15A-P4 (PTA-6507); 22C-P1 (PTA-6508); 2084 (NRRL B-500130); LSSA01 (NRRL-B-50104); BS27 (NRRL B-501 05); BS 18 (NRRL B-50633); and BS 278 (NRRL B-50634).

In another aspect, the present invention provides an animal feed comprising insects or insect meal treated with a polypeptide having protease activity. In one embodiment the treatment is a pre-treatment of insects or insect meals for use in an animal feed, i.e. the insects or insect meals are hydrolysed with a protease before insects or insect meal are in admixture with other components intended for addition to animal feed.

In a particular embodiment of a treatment process the protease(s) in question is affecting (or acting on, or exerting its hydrolyzing or degrading influence on) the insects or insect meal. To achieve this, the protein or protein source is typically suspended in a solvent, e.g. an aqueous solvent such as water, and the pH and temperature values are adjusted bases on the characteristics of the enzyme in question. For example, the treatment may take place at a pH-value at which the activity of the actual protease is at least 5%, 10%, 20%, 30%, 40%, 50%, 60%, 70%, 80%, or at least 90%. Likewise, for example, the treatment may take place at a temperature at which the activity of the actual protease is at least 5%, 10%, 20%, 30%, 40%, 50%, 60%, 70%, 80%, or at least 90%. The above percentage activity indications are relative to the maximum activities.

In another aspect, the present invention provides a method of degrading an arthropod exoskeleton, such as an insect exoskeleton comprising contacting said exoskeleton with a polypeptide having protease activity.

An arthropod is an invertebrate animal having an exoskeleton, a segmented body, and paired jointed appendages. Arthropods form the phylum Euarthropoda, which includes insects, arachnids, myriapods, and crustaceans. Arthropods are characterized by their jointed limbs and cuticle made of chitin, often mineralised with calcium carbonate. The arthropod body plan consists of segments, each with a pair of appendages. The rigid cuticle inhibits growth, so arthropods replace it periodically by moulting. Arthropods are bilaterally symmetrical and their body possess an exoskeleton.

In insects, the cuticle, which is primarily composed of chitin and cuticular proteins (CPs), covers the entire body surface, providing a stable environment that protects the insect by preventing excessive water evaporation and infection by exogenous pathogens. Chitin microfibrils are tightly associated with various cuticular proteins (Ephraim Cohen, in Encyclopedia of Insects (Second Edition), 2009). Surprisingly It was found that polypeptides having protease activity are significantly better than chitinases or glucanases at improving nutritional value of arthropod exoskeleton, including insects or insect meal.

In a preferred embodiment, polypeptides having protease activity are serine proteases. Without being bound by theory, serine proteases differ from other endopeptidases (cysteine endopeptidases, aspartic endopeptidases, metalloendopeptidases) because of the presence of the amino acid triads consisted of three amino acids: His 57, Ser 195 (hence the name “serine protease”) and Asp 102. The presence and the particular geometry of this triad can play an essential role in the cleaving of proteins containing CPs.

In a more preferred embodiment, the polypeptide having protease activity is an S1 or an S8 serine protease.

In another aspect, the present invention provides a method for improving nutritional value of insects or insect meal, comprising contacting the insects or insect meal with a polypeptide having protease activity.

The term improving nutritional value of an animal feed means improving the availability of nutrients in the feed. In this invention improving nutritional values refers in particular to improving solubilization of nitrogen from the insects or insect meal or increases digestible and/or soluble protein of the insects or insect meal. When nutritional value of the feed is increased, the growth rate and/or weight gain and/or feed conversion (i.e. the weight of ingested feed relative to weight gain) of the animal might be improved.

In one embodiment, the method of the present invention improves solubilization of nitrogen from the insects or insect meal or increases digestible and/or soluble protein of the insects or insect meal. According to the invention the protease and insects or insect meal can be fed to the animal before, after, or simultaneously with the diet. In a preferred embodiment, the insects or insect meal is comprised in an animal feed.

In another aspect, the present invention provides a method of preparing an animal feed comprising insects or insect meal, comprising contacting the insects or insect meal with a polypeptide having protease activity.

In a further aspect, the present invention provides a method for treating insect protein source or carbohydrate source including chitin, comprising the step of adding the polypeptide having protease activity to the insect protein source or carbohydrate source.

In a further aspect, the present invention provides use of a polypeptide having protease activity in an animal feed comprising insects or insect meal.

In one embodiment, the polypeptide having protease activity is a polypeptide having an acid stable protease activity. In a preferred embodiment, the polypeptide having protease activity is a serine protease, preferably an S1 or an S8 serine protease.

In another aspect, the present invention provides use of a polypeptide having protease activity in an animal feed comprising insects or insect meal;

in the preparation of a composition for use in an animal feed comprising insects or insect meal;

in the preparation of an animal feed additive for use in an animal feed comprising insects or insect meal;

for improving nutritional value of an animal feed comprising insects or insect meal;

for increasing digestible and/or soluble nitrogen in an animal feed comprising insects or insect meal;

for increasing the degree of hydrolysis of proteins and/or carbohydrates in animal diets comprising insects or insect meal; and/or

for the treatment of proteins and/or carbohydrates from insects or insect meal.

### EMBODIMENTS

1. An animal feed comprising an animal protein source and a polypeptide having protease activity, wherein the animal protein source comprises insects or insect meal and is free from fish protein.

2. An animal feed comprising an animal protein source and a polypeptide having protease activity, wherein the animal protein source is selected from the group comprising insects or insect meal and wherein the animal protein source is free from fish protein.

3. An animal feed comprising an animal protein source and a polypeptide having protease activity, wherein the animal protein source comprises insects or insect meal with the proviso that the animal feed is free from fish protein.

4. An animal feed comprising an animal protein source and a polypeptide having protease activity, wherein the animal protein source comprises insects or insect meal, with the proviso that the polypeptide having protease activity is not trypsin/pancreatin.

5. An animal feed comprising an animal protein source and a polypeptide having protease activity, wherein the animal protein source comprises insects or insect meal.

6. An animal feed according to embodiment 5, further comprising an animal protein source selected from the group consisting of meat meal, bone meal, poultry meal, blood, feather meal and seafood meal; and combinations thereof.

7. The animal feed according to any of embodiments 1 to 6, wherein the animal protein source comprises insect and/or insect meal and further comprises animal protein selected from blood meal, meat, bone meal, feather meal, and seafood meal (for example, shellfish, crab, lobster, shrimp meal, fish), and combinations thereof.

8. The animal feed according to any of embodiments 1 to 7, wherein the animal feed further comprises vegetable protein source.

9. The animal feed according to embodiment 8, wherein the vegetable protein source is selected from the group consisting of soybean, soybean meal, rapeseed, canola meal, sunflower, sunflower seed meal, cottonseed meal, DDGS, faba beans, peas, barley, wheat, rye, oat, maize (corn), rice, triticale, sorghum, palm oil cake; preferably, the vegetable protein source is selected from the group consisting of soybean, soybean meal, and maize (corn).

10. The animal feed according to any of embodiments 1 to 9, wherein the polypeptide having protease activity is a polypeptide having an acid stable protease activity.

11. The animal feed according to any of embodiments 1 to 10, wherein the polypeptide having protease activity is a serine protease, preferably an S1 or an S8 serine protease.

12. The animal feed according to any of embodiments 1 to 11, wherein the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;
(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and
(c) a fragment of the polypeptide of (a), or (b) that has protease activity.

13. The animal feed according to any of embodiments 1 to 12, wherein the polypeptide comprises or consists of SEQ ID NO: 1, the mature polypeptide of SEQ ID NO: 2, SEQ ID NO: 3, or SEQ ID NO: 4.

14. The animal feed according to any of embodiments 1 to 13, wherein the animal feed further comprises a protein source consisting of vegetable protein; preferably, the vegetable protein is selected from soybean, soybean meal, rapeseed, canola meal, sunflower, sunflower seed meal, cottonseed meal, DDGS, faba beans, peas, barley, wheat, rye, oat, maize (corn), rice, triticale, sorghum, palm oil cake.

15. The animal feed according to any of embodiments 1 to 14, wherein the animal feed comprises one or more further enzymes, preferably wherein the further enzymes are selected from the group comprising of amylases; phytases; xylanases; galactanases; alpha-galactosidases; phospholipases; and beta-glucanases; or any mixture thereof; more preferably the further enzymes are selected from the group comprising of amylases; phytases; xylanases; galactanases; alpha-galactosidases; and beta-glucanases; or any mixture thereof.

16. An animal feed comprising insects or insect meal treated with a polypeptide having protease activity.

17. The animal feed according to embodiment 16, wherein the polypeptide having protease activity is a polypeptide having an acid stable protease activity.

18. The animal feed according to any of embodiments 16 or 17, wherein the polypeptide having protease activity is a serine protease, preferably an S1 or an S8 serine protease.

19. The animal feed according to any of embodiments 16 to 18, wherein the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;
(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and
(c) a fragment of the polypeptide of (a) or (b) that has protease activity.

20. The animal feed according to any of embodiments 16 to 19, wherein the polypeptide comprises or consists of SEQ ID NO: 1, the mature polypeptide of SEQ ID NO: 2, SEQ ID NO: 3, or SEQ ID NO: 4.

21. The animal feed according to any of embodiments 1 to 20, wherein the insects are selected from the group of insect orders consisting of: Blattodea; Orthoptera; Diptera; Lepidoptera, and Coleoptera.

22. The animal feed according to embodiment 21, wherein the insects are selected from the group consisting of moths, butterflies; flies; beetles; crickets, and mealworms.

23. The animal feed according to any of embodiments 1 to 22, wherein animal is a mono-gastric animal, preferably a pig or poultry.

24. A method of degrading an arthropod exoskeleton, such as an insect exoskeleton comprising contacting said exoskeleton with a polypeptide having protease activity.

25. A method for improving nutritional value of insects or insect meal, comprising contacting the insects or insect meal with a polypeptide having protease activity.

26. The method according to embodiment 25, wherein the method improves solubilization of nitrogen from the insects or insect meal or increases digestible and/or soluble protein of the insects or insect meal.

27. The method according to embodiment 25 or 26, wherein the insects or insect meal is comprised in an animal feed.

28. A method of preparing an animal feed comprising insects or insect meal, comprising contacting the insects or insect meal with a polypeptide having protease activity.

29. The method according to embodiment 28, wherein the polypeptide having protease activity is a polypeptide having an acid stable protease activity.

30. The method according to embodiment 28 or 29, wherein the polypeptide having protease activity is a serine protease, such as an S1 or an S8 serine protease.

31. The method according to any of embodiments 28 to 30, wherein the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;
(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and
(c) a fragment of the polypeptide of (a) or (b) that has protease activity.

32. The method according to any of embodiments 28 to 31, wherein the polypeptide comprises or consists of SEQ ID NO: 1, the mature polypeptide of SEQ ID NO: 2, SEQ ID NO: 3, or SEQ ID NO: 4.

33. A method for treating insect protein source or carbohydrate source including chitin, comprising the step of adding the polypeptide having protease activity to the insect protein source or carbohydrate source.

34. The method according to embodiment 33, wherein the polypeptide having protease activity is a polypeptide having an acid stable protease activity.

35. The method according to embodiment 33 or 34, wherein the polypeptide having protease activity is a serine protease, such as an S1 or an S8 serine protease.

36. The method according to any of embodiments 33 to 35, wherein the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;
(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and
(c) a fragment of the polypeptide of (a) or (b) that has protease activity.

37. The method according to any of embodiments 33 to 36, wherein the polypeptide comprises or consists of SEQ ID NO: 1, the mature polypeptide of SEQ ID NO: 2, SEQ ID NO: 3, or SEQ ID NO: 4.

38. Use of a polypeptide having protease activity in preparation of an animal feed comprising insects or insect meal.

39. The use according to embodiment 38, wherein the polypeptide having protease activity is a polypeptide having an acid stable protease activity.

40. The use according to embodiment 39, wherein the polypeptide having protease activity is a serine protease, such as an S1 or an S8 serine protease.

41. Use of a polypeptide having protease activity
in an animal feed comprising insects or insect meal;
in the preparation of a composition for use in an animal feed comprising insects or insect meal;
in the preparation of an animal feed additive for use in an animal feed comprising insects or insect meal;
for improving nutritional value of an animal feed comprising insects or insect meal;
for increasing digestible and/or soluble nitrogen in an animal feed comprising insects or insect meal;
for increasing the degree of hydrolysis of proteins and/or carbohydrates in animal diets comprising insects or insect meal; and/or
for the treatment of proteins and/or carbohydrates from insects or insect meal.

42. Use according to embodiment 41, wherein the polypeptide having protease activity is a polypeptide having an acid stable protease activity.

43. The use according to embodiment 41 or 42, wherein the polypeptide having protease activity is a serine protease, such as an S1 or an S8 serine protease.

44. The use of the animal feed according to any of embodiments 41 to 43, wherein the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;
(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and
(c) a fragment of the polypeptide of (a) or (b) that has protease activity.

45. The use according to any of embodiments 41 to 44, wherein the polypeptide comprises or consists of SEQ ID NO: 1, the mature polypeptide of SEQ ID NO: 2, SEQ ID NO: 3, or SEQ ID NO: 4.

46. An animal feed comprising an animal protein source and a polypeptide having protease activity, wherein the animal protein source comprises insects or insect meal, wherein the polypeptide having protease activity is selected from the group consisting of:
(a) a polypeptide having at least 60%, e.g. at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 1, the mature polypeptide of SEQ ID NO: 2, SEQ ID NO: 3, or SEQ ID NO: 4;
(b) a variant of the polypeptide of SEQ ID NO: 1, the mature polypeptide of SEQ ID NO: 2, SEQ ID NO: 3, or SEQ ID NO: 4 comprising a substitution, deletion, and/or insertion of one or more (e.g. several) positions; and
(c) a fragment of the polypeptide of (a), or (b) that has protease activity.

47. The animal feed according to embodiment 46, wherein the polypeptide comprises or consists of SEQ ID NO: 1, the mature polypeptide of SEQ ID NO: 2, SEQ ID NO: 3, or SEQ ID NO: 4.

48. The animal feed according to embodiment 46 to 47, further comprising a protein source consisting of vegetable protein; preferably, comprising vegetable protein selected from soybean, soybean meal, rapeseed, canola meal, sunflower, sunflower seed meal, cottonseed meal, DDGS, faba beans, peas, barley, wheat, rye, oat, maize (corn), rice, triticale, sorghum, palm oil cake.

49. The animal feed according to any of embodiments 46 to 48, comprising one or more further enzymes selected from the group comprising of amylases; phytases; xylanases; galactanases; alpha-galactosidases; phospholipases; and beta-glucanases; or any mixture thereof; preferably comprising further enzymes selected from the group comprising of amylases; phytases; xylanases; galactanases; alpha-galactosidases; and beta-glucanases; or any mixture thereof.

The present invention is further described by the following examples that should not be construed as limiting the scope of the invention.

### EXAMPLES

### Materials and Methods

| | |
|---|---|
| protease | SEQ ID NO: 1 herein; also shown as |
| derived from | SEQ ID NO: 1 of WO 2001/058276 |
| Nocardiopsis | |
| sp. NRRL 18262 | |
| S8 protease from | mature polypeptide of SEQ ID NO: 2 herein; |
| Lysobacter | also shown as SEQ ID NO: 2 of WO 2019/043189 |
| IB-9374 | |
| S8 protease | SEQ ID NO: 3 herein; |
| from Bacillus | expressed and purified as described in Example |
| horneckiae | 1 of WO 015/091990 |
| a variant S8 | SEQ ID NO: 4 herein; |
| protease from | constructed, expressed and purified as described |
| Bacillus sp | in Example 1 of WO 2016/097354 |
| TY145 protease | |
| Glucanex ® | Lysing Enzyme from Trichoderma harzianum, |
| | comprising β-glucanase and chitinase |
| | activity; commercially available from |
| | Novozymes A/S |
| Chitinase-1 | GH18 chitinase-1 from Streptomyces griseus |
| Chitinase-2 | GH18 chitinase-2 from Streptomyces griseus |
| Streptomyces | Mature polypeptide of SEQ ID NO: 5 herein; |
| griseus GH18 | also shown as SEQ ID NO: 1 of WO2019236687. |
| chitinase | |

### pH-Stability Assay

Suc-AAPF-pNA (Sigma S-7388) was used for obtaining pH stability profiles.

Assay buffer: 100 mM succinic acid, 100 mM HEPES, 100 mM CHES, 100 mM CABS, 1 mM CaCl₂, 150 mM KCl, 0.01% Triton®X-100 adjusted to pH-values 2.0, 2.5, 3.0, 3.5, 4.0, 4.5, 5.0, 6 0, 7.0, 8.0, 9.0, 10.0 or 11.0 with HCl or NaOH.

Each protease sample (in 1 mM succinic acid, 2 mM CaCl₂, 100 mM NaCl, pH 6.0 and with an A₂₈₀ absorption >10) was diluted in the assay buffer at each pH value tested to A₂₈₀=1.0. The diluted protease samples were incubated for 2 hours at 37° C.

After incubation, protease samples were diluted in 100 mM succinic acid, 100 mM HEPES, 100 mM CHES, 100 mM CABS, 1 mM CaCl₂, 150 mM KCl, 0.01% Triton®X-100, pH 9.0, bringing the pH of all samples to pH 9.0.

In the following activity measurement, the temperature was 25° C.

300 μl diluted protease sample was mixed with 1.5 ml of the pH 9.0 assay buffer and the activity reaction was started by adding 1.5 ml pNA substrate (50 mg dissolved in 1.0 ml DMSO and further diluted 45× with 0.01% Triton®X-100) and, after mixing, the increase in A₄₀₅ was monitored by a spectrophotometer as a measurement of the (residual) protease activity.

The 37° C. incubation was performed at the different pH-values and the activity measurements were plotted as residual activities against pH. The residual activities were normalized with the activity of a parallel incubation (control), where the protease was diluted to A₂₈₀=1.0 in the assay buffer at pH 9.0 and incubated for 2 hours at 5° C. before activity measurement as the other incubations. The protease samples were diluted prior to the activity measurement in order to ensure that all activity measurements fell within the linear part of the dose-response curve for the assay.

### Example 1: In Vitro Solubilization of Nitrogen from 3 Types of Insect Meal

Black soldier fly, buffalo mealworm and cricket powder were ground down to the size of 0.5 mm with an Ultra Centrifugal Mill ZM 200 (commercially available from RETSCH) before any treatment.

A 250 mL beaker with 10 g of the different ground insects and 100 mL deionized water was placed on an IKA RCT magnetic stirrer with the settings 350 rpm and 40° C. The pH was measured with a Seven2Go pro pH-meter (commercially available from Mettler Toledo) and after reaching the set temperature, the pH was adjusted to pH 6 with either 1 M NaOH or 4 M HCl, depending upon the substrate used. Afterwards 4 mL of the solution was transferred into each well of a 24 deep well plate, resulting in 4 mL solution with approximately 400 mg insects in each well. The plate was sealed with sealing tape and finally closed with a lid and was placed on a microtiter stirrer for further 30 minutes with the settings 450 rpm and 40° C. Meanwhile the stock solutions for enzymes were prepared by weighing of 80 mg of either a protease derived from Nocardiopsis sp. NRRL 18262 or Glucanex (Novozymes A/S) and adding it in a 200 mL volumetric flask together with 200 mL enzyme dilution buffer and finally placed on a magnet stirrer. The composition of the enzyme dilution buffer was 0.025 g BSA (bovine serum albumin), 2.5 mL 1% Tween 20, and 250 mL 0.1 M Acetate buffer of pH 5, lastly the pH was adjusted to 6 with 1.5 mL 4M NaOH. Calculation of Glucanex® and the protease derived from Nocardiopsis sp. NRRL 18262 was made by mg enzyme product per kg diet. The chitinases (chitinase-1, chitinase-2) were diluted with enzyme dilution buffer till the concentration of purified enzyme was 0.4 mg/mL. The pH was measured before treatment was started. After preheating, 100 μL of enzyme dilution buffer (control) and 100 μL of the stock solutions of Glucanex®, the protease derived from Nocardiopsis sp. NRRL 18262 was added in 4 wells each, whereas chitinase-1 and chitinase-2 were added in 3 wells each. The placement of samples on the 24 deep well plate were random. Incubation time was 4 hours and approximately every 30 minutes the pH was measured. After incubation the plate was centrifuged with 4000 rpm for 10 minutes at 5° C. and the supernatant was removed from the plate with a plastic pipette carefully without the pellet and transferred into a new 24 deep well plate. Both 24 deep well plates were stored at −20° C. until further analysis. In order to identify any increase of nitrogen in the supernatant, a combustion analysis was made with the nitrogen analyser LECO FP628 by measuring the nitrogen content. The 24 deep well plates containing the supernatant were thawed and centrifuged with 4000 rpm for 10 minutes at 5° C., thereby removing any insoluble bioavailable matter. Afterwards 200 μL from each sample was transferred into a tin foil cup, and placed at 60° C. in an incubator overnight. The tin cups were not closed during incubation or analysis. After incubation the samples were combusted and results were analyzed. The results were shown in table 1.

TABLE 1
Normalized soluble nitrogen in supernatants from example
1. The values were an average of quadruplicates for
the Control, Glucanex ®, and the protease derived
from Nocardiopsis sp. NRRL 18262, and an average
of triplicates for chitinase-1 and chitinase-2.

| | | | Buffalo |
|---|---|---|---|
| | Cricket powder | Black soldier fly | mealworm |
| | Normalized | Normalized | Normalized |
| | soluble | soluble | soluble |
| Treatment | nitrogen | nitrogen | nitrogen |
| Control | 1.0000 | 1.0000 | 1.0000 |
| Glucanex ® | 0.9952 | 0.9791 | 1.0107 |
| Chintinase-1 | 0.9558 | 0.9480 | 0.9853 |
| Chitinase-2 | 0.9457 | 0.9753 | 0.9585 |
| Protease | 1.2838 | 1.0801 | 1.3644 |

On all three insect meals, the protease derived from Nocardiopsis sp. NRRL 18262 was significantly better at solubilizing nitrogen, compared to known chitinases.

### Example 2: In Vitro Solubilization of Nitrogen by Different Ddosages of Protease

Following the same procedure as in example 1, but with varying concentrations of the protease derived from Nocardiopsis sp. NRRL 18262. The results were shown in table 2.

TABLE 2
Normalized soluble nitrogen in supernatants from
example 2. The mean values were an average of
quadruplicates and afterwards normalized.

| | | | Buffalo |
|---|---|---|---|
| | Cricket powder | Black soldier fly | mealworm |
| | Normalized | Normalized | Normalized |
| | soluble | soluble | soluble |
| Treatment | nitrogen | nitrogen | nitrogen |
| Control | 1.0000 | 1.0000 | 1.0000 |
| 100 ppm protease | 1.0429 | 0.9981 | 1.0665 |
| 200 ppm protease | 1.0743 | 1.0057 | 1.1297 |
| 500 ppm protease | 1.1466 | 1.0245 | 1.2191 |
| 1000 ppm protease | 1.2123 | 1.0815 | 1.3084 |
| 2000 ppm protease | 1.3528 | 1.2824 | 1.4570 |

For all three insect meals, a clear dose response effect of the protease derived from Nocardiopsis sp. NRRL 18262 was seen, with higher dosages solubilizing more nitrogen.

### Example 3: In Vitro Solubilization of Nitrogen in Setup with Gastric Simulation Including Low pH and Pepsin

Furthermore, the enzymes were tested if they could function in a gastric simulation at pH 3 for 15 min including addition of pepsin, followed by incubation at pH 6 for 4 hours. 10,000 mg substrate was added to a 250 mL breaker together with 50 mL deionized water and placed on an IKA RCT with the settings 350 rpm and 40° C. The pH was measured and adjusted to pH 6 with either HCl/NaOH. In each of the wells of the 24 deep well plate 2 mL of the slurry was added and the plate was sealed with sealing tape and closed with the lid, placed on a microtiter stirrer for further preheating with the settings of 450 rpm and 40° C. Meanwhile, stock solutions of Glucanex® and the protease derived from Nocardiopsis sp. NRRL 18262 were made by adding 200 mg in 50 mL volumetric flask with enzyme dilution buffer and the three chitinases (chitinase-1, chitinase-2, and Streptomyces griseus GH18 chitinase) were diluted with enzyme dilution buffer till the concentration of purified enzyme was to 0.4 mg/mL. Furthermore, the stock solution for pepsin was prepared by mixing 63 mg pepsin and 183 mg calcium dichloride in 10 mL 0.1 M HCl. In addition, a 0.5 M sodium bicarbonate was made by dissolving 4200 mg in 100 mL deionized water and a 0.06 M sodium bicarbonate was made by taking 12 mL from the 0.5 M stock and diluting it in a 100 mL volumetric flask. The gastric simulation was started by adding 250 μL 1 M HCl, 300 μL 1 M HCl, and 300 μL HCl to each well of CP (Cricket powder), BSF (Black soldier fly), and BMW (Buffalo mealworm), respectively, followed by 100 μL from the pepsin stock solution. Incubation time was 15 minutes at pH 3. The pH was measured before, just after addition and 10 minutes in the incubation time. After the first 15 minutes 500 μL, 1200 μL, and 700 μL of 0.5 M sodium bicarbonate was added to each well of CP, BSF, and BWM, respectively, and with further 30 minutes incubation time. Lastly 1150 μL, 400 μL, and 900 μL of 0.06 M sodium bicarbonate were added to each well of CP, BSF, and BWM, respectively, followed by 100 μL enzyme dilution buffer, control, and 100 μL of the five different enzyme stock solutions, in a replication of four. Incubation time with treatment was four hours. After incubation the 24 deep well plates were centrifuged with 4000 rpm for 10 minutes at 5° C., and the supernatant was removed carefully to another 24 deep well plate, which was stored at −20° C. After incubation the plate was centrifuged with 4000 rpm for 10 minutes at 5° C. and the supernatant was removed from the plate with a plastic pipette carefully without the pellet and transferred into a new 24 deep well plate. Both 24 deep well plates were stored at −20° C. until further analysis. In order to identify any increase of nitrogen in the supernatant, a combustion analysis was made with LECO FP628 by measuring the nitrogen content. The 24 deep well plates containing the supernatent were thawed and centrifuged with 4000 rpm for 10 minutes at 5° C., thereby removing any insoluble bioavailable matter. Afterwards 200 μL from each sample was transferred into a tin foil cup, and placed at 60° C. in an incubator overnight. The tin cups were not closed during inbucation or analysis. After incubation the samples were combusted and results were analyzed. The results were shown in table 3.

TABLE 3
Normalized soluble nitrogen in supernatants from example 3. The
values were a normalization of the average of quadruplicates.

| | | | Buffalo |
|---|---|---|---|
| | Cricket powder | Black soldier fly | mealworm |
| | Normalized | Normalized | Normalized |
| | soluble | soluble | soluble |
| Treatment | nitrogen | nitrogen | nitrogen |
| Control | 1.0000 | 1.0000 | 1.0000 |
| Chintinase-1 | 0.9819 | 1.0068 | 1.0042 |
| Chintinase-2 | 0.9818 | 1.0226 | 1.0049 |
| Streptomyces | 0.9862 | 1.0138 | 1.0185 |
| griseus GH18 | | | |
| chitinase | | | |
| Glucanex ® | 0.9781 | 1.0021 | 1.0201 |
| Protease | 1.2883 | 1.0548 | 1.4180 |

On all three insect meals, the protease derived from Nocardiopsis sp. NRRL 18262 was significantly better at solubilizing nitrogen in the gastric step simulation, compared to known chitinases.

### Example 4: In Vitro Solubilization of Nitrogen in Setup with Gastro Intestinal Tract Simulation Including low pH, Pepsin and Pancreatin

The gastro intestinal trach simulation was similar to the gastric simulation performed in example 3, however this time pancreatin and bile salt was dissolved in the 0.06 M sodium bicarbonate stock solution, for a concentration of 15.34 mg/mL bile salt and 0.696 mg/mL pancreatin for cricket powder, 44.1 mg/mL bile salt and 2 mg/mL pancreatin for black soldier fly, and 25.2 mg/mL bile salt and 1.14 mg/mL pancreatin for Buffalo mealworm.

TABLE 4
Normalized soluble nitrogen in supernatants from example 4. The
values were a normalization of the average of quadruplicates.

| | | | Buffalo |
|---|---|---|---|
| | Cricket powder | Black soldier fly | mealworm |
| | Normalized | Normalized | Normalized |
| | soluble | soluble | soluble |
| Treatment | nitrogen | nitrogen | nitrogen |
| Control | 1.0000 | 1.0000 | 1.0000 |
| Chitinase-1 | 0.9860 | 1.0295 | 1.0006 |
| Chitinase-2 | 0.9781 | 1.0284 | 1.0037 |
| Streptomyces | 0.9988 | 0.9893 | 1.0121 |
| griseus GH18 | | | |
| chitinase | | | |
| Glucanex ® | 0.9648 | 0.9991 | 0.9993 |
| Protease derived | 1.0706 | 1.0798 | 1.1190 |
| from Nocardiopsis | | | |
| sp. NRRL 18262 | | | |

On all three insect meals, the protease derived from Nocardiopsis sp. NRRL 18262 was significantly better at solubilizing nitrogen in the gastro intestinal tract simulation, compared to known chitinases.

### Example 5: Solubilization of Nitrogen from 3 Types of Insect Meals with Three S8 Proteases and Protease Derived from Nocardiopsis sp. NRRL 18262

5 g milled insect meal was weighed in a beaker and 50 ml MilliQ water was added. This slurry was pre-incubated for 15 min at 40° C. 2 ml of the slurry was added to each well on a 24 deep well plate, which was pre-incubated on a microtiter stirrer for 15 min at 40° C. The enzymes were diluted in enzyme dilution buffer (see description of enzyme dilution buffer in example 1) and 100 ul of enzyme or enzyme dilution buffer for control were added to the wells. The enzymes were dosed at 200 ppm. The samples were incubated at pH 6 for 2 hours at 40° C. in 4 replicates per treatment. After incubation the plates were centrifuged (3000 rpm, 10 min, 5° C.) and the supernatant was collected and frozen. In order to identify any increase of protein in the supernatant OPA (O-phthaldialdehyde) absorbance spectroscopy measurements were preformed. As the samples were thawed the OPA reagent stock solution was prepared by the following steps: In a 150 mL volumetric bottle 1.01 g hydrogen carbonate, 0.8586 g sodium carbonate decahydrate, and 150 mg sodium dodecyl sulphate were added and ⅔ of the bottle was filled with deionized water, left on magnet stirring for dissolving. Meanwhile, approximately 120 mg O-Phthaldialdehyd was added to a greiner tube, which was wrapped in tinfoil, dissolved in 3 ml 99.9% ethanol by being placed on a vortex mixer for 2 minutes. Afterwards 3 mL was added to the volumetric bottle and the bottle was wrapped in tinfoil. Lastly, 120 mg Di-Thiothreitol was added and the bottle was filled up for the final total volume of 150 mL, and placed on a magnet stirring. Standard solution was prepared by dissolving 50 mg L-serine in 500 mL deionized water, and stored at 5° C. The samples were prepared by centrifuging 300 μL supernatant in eppendorf tube for 1 min at 14,000 rpm at 5° C., and diluting samples from CP and DBW 1:5 and 1:8 from BSF with deionized water for the total volume of 400 μL. Afterwards, the diluted samples were shaken and 300 μL from each sample were transferred in wells of PALL Corporation Acroprep advance 96 filter platewhich was fixed on a 96 microwell plate. The new plate was centrifuged with 2700 rpm for 10 min at 5° C., after centrifugation the filter was removed and the 96 microwell plate was sealed with sailing tape and shaken with 650 rpm for 1 min. Afterwards the plate was placed in the Hamilton starlet which was further diluting 1:10 and mixing the samples with the OPA reagent stock solution. When a new plate was made by the Hamilton it was sealed with sealing tape and shaking with 650 rpm for 1 min before analysing the absorbance. The adsorbance spectrum was recorded with the wavelength of 340 nm.

TABLE 5
Normalized absorption in OPA assay corresponding to the number
of free amino ends in the supernatant for example 5.

| | | Black | Buffalo |
|---|---|---|---|
| | Cricket powder | soldier fly | mealworm |
| | Normalized | Normalized | Norrmalized |
| | absorbance | absorbance | absorbance |
| Treatment | in OPA assay | in OPA assay | in OPA assay |
| Control | 1.0000 | 1.0000 | 1.0000 |
| S8 protease from | 1.1392 | 1.2319 | 1.2293 |
| Lysobacter | | | |
| IB-9374 | | | |
| S8 protease | 1.1697 | 1.1924 | 1.0514 |
| from Bacillus | | | |
| horneckiae | | | |
| a variant S8 | 1.1923 | 1.3883 | 0.9983 |
| protease from | | | |
| Bacillus sp | | | |
| TY145 protease | | | |
| Protease | 1.2167 | 1.6139 | 1.4336 |
| derived from | | | |
| Nocardiopsis | | | |
| sp. NRRL 18262 | | | |

The protease derived from Nocardiopsis sp. NRRL 18262 and the 3 tested S8 proteases increased the amount of free amino ends in the supernatant after incubation.

### Example 6: The Efficacy of a Variant S8 Protease from Bacillus sp TY145 Protease on Broiler Performance

A feeding study with male broilers was conducted to investigate the efficacy of protease on broiler performance. Three isonitrogeneous diets were used within three feeding periods from 0-7 (starter), 8-21 (grower) and 22-35 (finisher) days of age. The positive control (PC) was a diet fully optimized within the growth period, with high protein digestibility, and the negative controls (NC80 and NC70) were formulated with poor protein quality, lower protein digestibility and reduced energy content.

### Materials and Methods

At the day of hatching, 2700 male day-old Ross 308 broiler chickens were randomly assigned in groups of 25 chickens to the experimental pens (˜3 sq.m.) equipped with a bell drinker and a round feeder. Birds were vaccinated against Newcastle disease and Gumboro at the age of 18 days. Three basal diets with decreasing protein quality and digestibility were formulated according to the breeders recommendations in three phases and consisted mainly of corn and soybean meal. Soybean meal was substituted with corn DDGS in the diets where protein quality was reduced. All diets were supplemented with phytase RONOZYME® HiPhos (10000 FYT, commercially available from DSM) and a coccidiostat. All diets contained titanium dioxide at 0.5% on top of given diet formulations. All diets were fed as control diets (no enzyme added) or supplemented with a variant S8 protease from Bacillus sp TY145 protease. Each of the six treatments was fed to 18 replicate pens of 25 male broilers. Body weight was recorded at placement and in the end of each growing period. Body weight gain (BW gain), feed consumption, and feed conversion (all mortality corrected) were calculated. The feed conversion ratio (FCR) was calculated as:

FCR[kg feed/kg gain]=total feed consumption of a pen divided by total BW gain of that pen (total BW gain=total BW at the end+weight of removals and losses−total BW at the beginning).

Feed intake (FI) per bird was calculated as:

FI [g feed/day]=FCR*Body weight gain

TABLE 6
Diets

| | STARTER | | | GROWER | |
|---|---|---|---|---|---|
| | Starter_PC | Starter_NC80 | Starter_N70 | Grower_PC | Grower_NC80 |
| Corn NL_Prot | 499.61 | 497.41 | 498.91 | 528.06 | 524.41 |
| Corn DDGS NL_prot | | 43.23 | 69.01 | | 51.39 |
| Soya O/C 48.5% | 396.00 | 370.00 | 351.00 | 363.00 | 330.00 |
| NL_prot | | | | | |
| Limestone | 18.00 | 18.00 | 18.00 | 16.00 | 16.00 |
| Monocalcium Phos | 10.30 | 10.00 | 10.00 | 9.00 | 8.50 |
| Salt | 3.00 | 2.00 | 1.00 | 3.00 | 2.00 |
| Sodium Bicarbonate | 1.84 | 1.26 | 2.14 | 1.60 | 1.65 |
| Soy Oil | 58.52 | 44.07 | 36.89 | 68.56 | 53.27 |
| DL Methionine | 3.90 | 4.60 | 3.20 | 3.30 | 2.80 |
| L Threonine | 1.10 | 0.97 | 0.95 | 0.70 | 0.60 |
| L Valine | 0.25 | 0.40 | 0.00 | | |
| Lysine HCL | 2.10 | 2.00 | 3.00 | 1.30 | 3.40 |
| Broiler premix | 5.00 | 5.00 | 5.00 | 5.00 | 5.00 |
| Phytase 10000 FYT | 0.15 | 0.15 | 0.05 | 0.15 | 0.05 |
| Monteban G100 | 0.60 | 0.60 | 0.60 | 0.60 | 0.60 |
| Coccidiostats | | | | | |

| | GROWER | FINISHER | | |
|---|---|---|---|---|
| | Grower_NC70 | Finisher_PC | Finisher_NC80 | Finisher_NC70 |
| Corn NL_Prot | 528.01 | 580.41 | 578.41 | 570.41 |
| Corn DDGS NL_prot | 80.61 | | 52.98 | 84.12 |
| Soya O/C 48.5% | 305.00 | 304.00 | 273.00 | 257.00 |
| NL_prot | | | | |
| Limestone | 17.00 | 17.00 | 15.00 | 15.00 |
| Monocalcium Phos | 8.00 | 8.00 | 7.50 | 7.00 |
| Salt | 1.00 | 2.00 | 2.00 | 2.00 |
| Sodium Bicarbonate | 2.61 | 4.90 | 1.39 | 0.97 |
| Soy Oil | 45.29 | 68.19 | 55.01 | 48.54 |
| DL Methionine | 2.80 | 4.00 | 4.00 | 4.00 |
| L Threonine | 0.66 | 1.82 | 1.50 | 1.50 |
| L Valine | | 2.00 | 1.20 | 1.50 |
| Lysine HCL | 3.00 | 1.70 | 2.50 | 2.50 |
| Broiler premix | 5.00 | 5.00 | 5.00 | 5.00 |
| Phytase 10000 FYT | 0.05 | 0.05 | 0.05 | 0.05 |
| Monteban G100 | 0.60 | 0.60 | 0.60 | 0.60 |
| Coccidiostats | | | | |

TABLE 7
Analysed nutrient composition

| | | | | | | | | |
|---|---|---|---|---|---|---|---|---|
| | AME_{N} | DM | CP | CF | EE | Ash | Starch | Sugars |
| Diet² | MJ/kg | % | % | % | % | % | % | % |

Starter

| | | | | | | | | |
|---|---|---|---|---|---|---|---|---|
| PC | 13.0 | 89.7 | 22.9 | 2.7 | 8.8 | 6.0 | 35.8 | 4.0 |
| PC + | 13.0 | 89.8 | 23.3 | 2.5 | 8.2 | 5.9 | 36.1 | 3.9 |
| protease | | | | | | | | |
| NC80 | 12.8 | 89.6 | 23.1 | 2.7 | 7.9 | 6.1 | 36.0 | 3.8 |
| NC80 + | 12.6 | 89.8 | 22.9 | 2.7 | 7.3 | 5.7 | 36.1 | 4.0 |
| protease | | | | | | | | |
| NC70 | 12.6 | 89.6 | 22.9 | 2.5 | 7.3 | 5.5 | 36.4 | 3.8 |
| NC70 + | 12.7 | 89.9 | 23.2 | 2.5 | 7.6 | 6.3 | 35.9 | 3.7 |
| protease | | | | | | | | |

Grower

| | | | | | | | | |
|---|---|---|---|---|---|---|---|---|
| PC | 13.4 | 89.3 | 21.3 | 2.2 | 9.3 | 5.7 | 38.3 | 3.6 |
| PC + | 13.4 | 89.5 | 21.5 | 2.7 | 9.5 | 5.4 | 37.9 | 3.5 |
| protease | | | | | | | | |
| NC80 | 13.0 | 89.1 | 21.1 | 3.0 | 8.6 | 4.9 | 37.7 | 3.5 |
| NC80 + | 12.8 | 89.7 | 22.1 | 2.7 | 8.1 | 5.5 | 37.0 | 3.3 |
| protease | | | | | | | | |
| NC70 | 12.6 | 89.5 | 21.7 | 2.5 | 8.2 | 5.4 | 35.7 | 3.3 |
| NC70 + | 12.8 | 89.8 | 21.4 | 2.2 | 8.4 | 5.8 | 36.7 | 3.3 |
| protease | | | | | | | | |

Finisher

| | | | | | | | | |
|---|---|---|---|---|---|---|---|---|
| PC | 13.7 | 89.7 | 20.1 | 2.2 | 9.6 | 5.5 | 41.0 | 3.6 |
| PC + | 13.5 | 89.6 | 20.0 | 2.7 | 9.6 | 5.6 | 40.6 | 3.5 |
| protease | | | | | | | | |
| NC80 | 13.5 | 89.7 | 20.1 | 2.7 | 9.4 | 5.5 | 40.0 | 3.3 |
| NC80 + | 13.5 | 89.6 | 19.8 | 2.5 | 9.2 | 5.5 | 41.5 | 3.6 |
| protease | | | | | | | | |
| NC70 | 13.0 | 89.6 | 19.9 | 2.7 | 8.5 | 5.4 | 40.1 | 3.3 |
| NC70 + | 13.1 | 89.4 | 19.9 | 2.7 | 8.5 | 5.4 | 40.0 | 3.3 |
| protease | | | | | | | | |

DM—Dry matter,
CP—Crude protein,
CF—Crude fiber,
EE—Crude fat Ether extract

### Results and Conclusions

The effect of the protease supplementation resulted in an improvement of FCR and BW gain. In the finisher period the modelled data indicated that the BW gain was 1663 g and 1690 g for the birds fed the control diets and for the birds fed the diets supplemented with a protease, respectively. In the finisher period the modelled data indicated that the FCR was 1.501 and 1.483 for the birds fed the control diets and the diets supplemented with a protease, respectively. The final BW gain was 2797 g and 2825 g for the birds fed the control and the protease-supplemented diet, respectively. The final BW gain was 1.355 and 1.345 g for the birds fed the control and the protease-supplemented diet, respectively.

As previously demonstrated (US20200196633A1), the protease is able to hydrolyze proteins in corn/SBM-diet. The good performance of protease in hydrolyzing proteins from corn/SBM-diet resulted in the lower FCR and higher BWG in animal trial. As demonstrated in Examples 1-5, the proteases are able to hydrolyze proteins of different insects and insect meals and therefore, the proteases are expected to have a good performance in improving FCR and BWG parameters in animal trials.

1-19. (canceled)

20. A method comprising contacting an arthropod exoskeleton or a fragment thereof with a polypeptide having protease activity, said polypeptide selected from the group consisting of:
the polypeptide of SEQ ID NO: 1 and variants and fragments thereof;
the polypeptide of SEQ ID NO: 2 and variants and fragments thereof;
the polypeptide of SEQ ID NO: 3 and variants and fragments thereof; and
the polypeptide of SEQ ID NO: 4 and variants and fragments thereof.

21. The method of claim 20, wherein said arthropod skeleton is an insect exoskeleton.

22. The method of claim 20, wherein said polypeptide comprises the polypeptide sequence of SEQ ID NO: 1.

23. The method of claim 20, wherein said polypeptide comprises the polypeptide sequence of SEQ ID NO: 2.

24. The method of claim 20, wherein said polypeptide comprises the polypeptide sequence of SEQ ID NO: 3.

25. The method of claim 20, wherein said polypeptide comprises the polypeptide sequence of SEQ ID NO: 4.

26. The method of claim 20, wherein said arthropod exoskeleton or fragment thereof is comprised within an insect meal.

27. The method of claim 20, wherein said arthropod exoskeleton or fragment thereof is comprised within an animal feed.

28. The method of claim 20, wherein said arthropod exoskeleton or fragment thereof is comprised within a pelleted animal feed.


SEQUENCE LISTING (5 sequences)

SEQ ID NO: 1 - protein, 188 aa, Nocardiopsis sp. NRRL 18262
ADIIGGLAYTMGGRCSVGFAATNAAGQPGFVTAGHCGRVGTQVTIGNGRGVFEQSVFPGNDAAFVRGTSNFTLTNLVSRYNTGGYAAVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 2 - protein, 465 aa, Lysobacter sp. IB-9374
MSSPSMLRLRRHALAAAVLCLIAAPAFADERVDLSGLKADAPVDGFLVSYAADVPKAAAFQRDLDIALSSLGRKDLKIQHTRTISTGAELIEVNRALDVVEAATLMKSIAADPRVRFVEPNARFQALLTPNDPLYSQQWGLSGTYGIRANTAWDNGYQGQGKIIAVVDTGITDHPDLLANRTSPLGYDFISNATTANDGNGRDSDPHDPGDWTTAGQCGLGQPARNSSWHGTHVSGIAAGVTNNSTGIAGTAFQAKILSARVLGRCGGTLADIADAITWASGGTVSGVPAVGANKATVINMSLGGGGACSASSAMQVAITGAVSRGVTVVVAAGNSNADASGFQPASCANVINVGATTSAGVRASFSNYGSLVDVAAPGQTILSTLNAGTTSPGAFNYVNYNGTSMAAPFVAGVVALMQSKPGTDLTPAQVEATIKNTASPFASPQSPSLGTGIVNADAATDATP

SEQ ID NO: 3 - protein, 314 aa, Bacillus horneckiae
  mat_peptide (1)..(314)
EVTATPSTQTPWGIKSIYNDQSITKTTGGSGIKVAVLDTGVHTGHIDLAGSSEQCKDFTQSNPLVNGSCTDRQGHGTHVAGTVLAHGGSDGQGVYGVAPQAKLWAYKVLGDNGSGYSDDIAAAIRHVADEASRTGSKVVINMSLGSSGKDSLIASAVDYAYGKGVLIVAAAGNSGSGSNTIGYPAALVNAVAVAALENVQQNGTYRVANFSSRGNPATAGDFRIQERDVEVSAPGASVESTWYNGGYNTISGTSMATPHVAGLAAKIWSSNSSLSHSQLRTELQNRAKVYDIKGGIGAGTGDDYASGFGYPRVK

SEQ ID NO: 4 - protein, 311 aa, Artificial Sequence
  Protein Engineered variant
  mat_peptide (1)..(311)
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 5 - protein, 423 aa, Streptomyces griseus
MFKLHRPRARLRALAAAACTAALGATLLGVAGMSPAGATPAAQSAASPAAAEAAPTAAGDKVIGYFTNWGTYDRNYHVKNIETSGSADKLTHINYAFGNVIGGKCTIGDSYADYEKAYTADQSVDGVADTWDQPLRGNFNQLRKLKKLHPDLKILWSFGGWTWSGGFGQAAQNPAAFAKSCRDLVEDPRWADVFDGIDIDWEYPNACGLTCDTSGRDAYGNLLGELRKSFGTDLVTSAITADGSDGGKIDAVDYAGAAKHLDWYLPMTYDFFGAWEAKGPTAPHSPLTSYPGVPTEGFNSDAAISKLKSLGIPPEKLLLGIGFYGRGWTGVTRSEPGGSATGAAAGTYEAGIEDYRVLKNSCPATGKVAGTAYAHCGTDWWSYDTPETIGSKMNYKNEQGLGGTFFWELSGDTGNGELIKAIR